(19) United States
(12) Patent Application Publication
(10) Pub. No.: US 2001/0041346 A1
(43) Pub. Date: Nov. 15, 2001

(54) METHOD FOR ASSAYING COMPOUNDS AFFECTING SMOOTH MUSCLE CONTRACTILE STATE

(76) Inventor: MICHAEL E. MENDELSOHN, WELLESLEY, MA (US)

Correspondence Address: PAUL T CLARK, CLARK & ELBING LLP, 176 FEDERAL STREET, BOSTON, MA, 02110

(21) Appl. No.: 09/354,354

(22) Filed: Jul. 14, 1999

(51) Int. Cl. G01N 33/573

(52) U.S. Cl. 435/7.4

(57) ABSTRACT

The invention provides a method for assaying compounds that affect smooth muscle cell function by determining the interaction between a cyclic GMP-dependent protein kinase, and a myosin phosphatase myosin-binding subunit.

### STATEMENT AS TO FEDERALLY SPONSORED RESEARCH

The invention was made, in part, with funding from the National Institutes of Health, grants HL09330 and HL55309. The government may have certain rights in the invention.

### FIELD OF THE INVENTION

The invention relates to assays for compounds affecting smooth muscle contraction and relaxation.

### BACKGROUND OF THE INVENTION

Smooth muscle cells play a critical role in maintaining the physiological state of many organs in the body, including the vascular, respiratory, and gastrointestinal systems. Smooth muscle cell contractile state determines vascular tone and many ischemic, hypertensive, and vascular diseases arise, in part, from abnormalities in smooth muscle cell function. Smooth muscle cell dysfunction, therefore, plays a key role in the pathogenesis of a variety of diseases, including atherosclerosis, arterial hypertension, coronary artery disease, cardiovascular disease, cerebrovascular disease including stroke and migraine, disorders of the gastrointestinal system and disorders of sexual organ function.

It would be useful to have compounds that are capable of affecting the contractile state of smooth muscle. This would enable the identification of drugs suitable for the treatment, diagnosis, or prevention of diseases associated with smooth muscle cell dysfunction.

### SUMMARY OF THE INVENTION

The invention provides assays for the identification of compounds that affect the contractile state of smooth muscle cells.

In the first aspect, the invention provides a method of assaying whether a test compound affects the contractile state of smooth muscle cells, the method including providing a system including a cyclic GMP-dependent protein kinase, and a myosin phosphatase myosin-binding subunit, under conditions in which the kinase can interact with the subunit; contacting the test compound with the system; and determining whether the test compound affects the interaction between the kinase and the subunit as an indication of the ability of the test compound to affect the contractile state of smooth muscle cells.

In preferred embodiments of the invention, the cyclic GMP-dependent protein kinase may be full length cGK1α, or mutants of cGK1α, such as cGK₁₋₅₉ or a leucine zipper mutant, and the myosin phosphatase myosin-binding subunit may be full length or a fragment, such as AL9.

In other preferred embodiments of the invention, the smooth muscle cell may be a vascular smooth muscle cell.

In other preferred embodiments of the invention, determining whether the test compound affects the interaction between the kinase and the phosphatase subunit may be done by measuring any combination of the following indices: phosphatase activity, kinase activity, myosin light chain phosphorylation state, vascular tone in a ring or strip bioassay, vascular smooth muscle cell length, or sub-cellular location of the kinase or the phosphatase.

In other preferred embodiments of the invention, the interaction may be determined in vitro or in vivo, using protein interaction methods such as the yeast two-hybrid system, immunoprecipitation, GST-fusion protein assays, fluorescence spectroscopy, or any combination of protein interaction assays known in the art.

In other preferred embodiments of the invention, the system may include compounds that may activate the kinase such as: non-nitrate vasodilators; nitrovasodilators, for example, nitric oxide or nitrates; cGMP analogues, for example, 8-bromo-cyclic GMP; or compounds that alter the level of cGMP, such as phosphodiesterase inhibitors, for example, Viagra™.

By “test compound” is meant any chemical compound, be it naturally-occurring or artificially-derived. Test compounds may include, for example, peptides, polypeptides, synthesized organic molecules, naturally occurring organic molecules, and nucleic acid molecules.

By “affects” is meant changes, either by increase or decrease.

By “contacting” is meant to submit an animal, cell, lysate, or extract derived from a cell, or molecule derived from a cell, to a test compound.

By “contractile state” is meant a determination of smooth muscle cell contraction or relaxation as measured by PP1M phosphatase activity, myosin light chain phosphorylation state, the tone of a vascular ring or strip in a vascular bioassay system or in an in vivo model, the length of individual smooth muscle cells, and related indices.

By “determining” is meant analyzing the effect of a test compound on the test system. The readout of the analysis may be altered protein interaction and/or altered phosphatase activity, as well as measures of contractile state of smooth muscle cells. The means of analyzing may include, for example, yeast two-hybrid assays, GST fusion protein interaction, immunoprecipitation, fluorescence spectroscopy, and other methods known to those skilled in the art.

By “myosin binding subunit” is meant a polypeptide chain that interacts with the other polypeptide chains of a myosin phosphatase to form a multi-subunit protein complex as described in, for example, Alessi et al., Eur. J Biochem., 210: 1023-1035, 1992; Hirano et al., J. Biol. Chem. 272: 3683, 1997; Johnson, et al., Eur. J. Biochem., 239: 317, 1996; and Shimizu, et al. J. Biol. Chem. 269: 30407, 1994.

The invention provides a means of identifying test compounds that affect smooth muscle contractile state. This is particularly useful since smooth muscle cell dysfunction has been associated with a variety of conditions associated with sexual dysfunction, disorders of gastrointestinal motility, and vascular, hypertensive, and ischemic diseases. Thus, compounds that affect smooth muscle contractile state may be used in therapy, prevention, or diagnosis of such diseases.

Other features and advantages of the invention will be apparent from the following detailed description and from the claims.

### BRIEF DESCRIPTION OF THE DRAWINGS

FIG. 1A is a photograph of yeast strain Y190, transformed with cGKIα/GalDB (cGK-BD) or GalDB alone (pC97-BD), in combination with AL9/GalAD (AL9-AD) or GalAD alone (pC86-AD), and plated on YPD and Sc-His plates. Also shown is a colony lift β-galactosidase assay (LacZ) of yeast from the YPD plate.

FIG. 1B is a photograph of yeast strain Y190 transformed with AL9/GalAD (AL9-AD) in combination with cGKIα/GalDB truncation mutants (BD fusions) and plated on YPD and Sc-His plates. Also shown is a colony lift β-galactosidase assay (LacZ) of yeast from the YPD plate.

FIG. 1C is a summary of HIS and β-Gal reporter activation for the cGK1α/GalDB truncation mutants, cotransformed in Y190 with AL9/GalAD, as shown in FIG. 1B.

FIG. 1D is a summary of site-directed mutagenesis experiments in which selected leucine and isoleucine residues in cGKIα₁₋₅₉ were mutated to either alanine (A) or proline (P).

FIG. 1E is a schematic diagram showing binding of cGKIα to the MBS of PP1M.

FIG. 2A is an immunoblot showing the specific interaction between MBS fragment AL9 and cGKIα in human vascular smooth muscle cells.

FIG. 2B is an immunoblot showing the interaction of peptides derived from cGK isoforms with MBS from human saphenous vein smooth muscle cell lysates.

FIG. 3A is an immunoblot of lysates from cultured saphenous vein smooth muscle cells, immunoprecipitated with control or anti-cGKIα antibody, and immunoblotted for MBS.

FIG. 3B is an immunoblot of lysates from cultured saphenous vein smooth muscle cells, immunoprecipitated with control or anti-MBS antibody, and immunoblotted for cGKIα (cGK).

FIG. 3C is a bar graph showing the association of PP1 phosphatase activity with cGKIα.

FIG. 3D is a bar graph showing the activation of PP1 phosphatase activity in MBS immunopellets by 8-bromo-cGMP.

FIG. 3E is an autoradiograph showing in vitro phosphorylation of proteins in a MBS immunopellet by cGKIα.

FIG. 3F is an autoradiograph showing in vitro MBS phosphorylation assays without cGK (Ctl), or with constitutively active cGKIα (cGK-CA) or full length cGKIα (cGK-FL). Control phosphorylations with the general cGKIα substrate histone F2b are shown in the lower panel.

FIG. 4A is a photograph showing vascular smooth muscle cells immunostained with anti-cGKIα antibody.

FIG. 4B is a photograph showing vascular smooth muscle cells immunostained with anti-MBS antibody.

FIG. 4C is a photograph showing vascular smooth muscle cells immunostained with anti-cGKIα and anti-MBS antibody. Superimposition of the images of FIG. 4A and FIG. 4B.

FIG. 4D is a photograph showing vascular smooth muscle cells permeabilized prior to fixation to reveal actin-myosin stress fibers and immunostained with anti-cGKIα antibody.

FIG. 4E is a photograph showing vascular smooth muscle cells permeabilized prior to fixation to reveal actin-myosin stress fibers and immunostained with anti-MBS antibody.

FIG. 4F is a photograph showing vascular smooth muscle cells permeabilized prior to fixation to reveal actin-myosin stress fibers and immunostained with anti-cGKIα and anti-MBS antibody. Superimposition of the images of FIG. 4D and FIG. 4E.

FIG. 5 is a bar graph showing the effect of cGMP on myosin light chain phosphorylation in native vascular smooth muscle cells and following disruption of the cGK-MBS interaction.

### DETAILED DESCRIPTION OF THE INVENTION

Smooth muscle contraction and relaxation are regulated, largely, by changes in intracellular calcium levels and by calcium-independent effects on the activity of myosin light chain phosphatase (PP1M) (Kamm et al., Annu. Rev. Pharmacol. Toxicol. 25: 593, 1985; Hartshorne, in Physiology of the Gastrointestinal Tract, D. R. Johnson, Ed. (Raven, New York, ed. 2, 1987, vol. 1, pp. 423-482; Filo et al., Science 147: 1581, 1965; Somlyo et al., FASEB J. 3: 2266, 1989; Somlyo et al., Nature, 372: 231-236, 1994). An increase in intracellular calcium causes smooth muscle cell contraction by activation of the calcium/calmodulin-dependent myosin light chain kinase, which phosphorylates myosin light chain and activates the contractile myosin ATPase. A decrease in intracellular calcium results in inactivation of myosin light chain kinase, leading to dephosphorylation of myosin light chain by the myosin light chain phosphatase, PP1M (Somlyo et al., Nature, 372: 231-236, 1994). PP1M is a trimeric protein, comprising a 130 kD regulatory myosin binding subunit (MBS), a 37 kD catalytic subunit (PP1c), and a 20 kD subunit (M20) (Alessi et al., Eur. J. Biochem., 210: 1023-1035, 1992; Hirano et al., J Biol. Chem. 272: 3683, 1997; Johnson, et al., Eur. J. Biochem., 239: 317, 1996; Shimizu, et al. J Biol. Chem. 269: 30407, 1994).

The sensitivity of the smooth muscle contractile apparatus to calcium is modulated by intracellular messengers that alter PP1M activity. Contractile agonists, acting through signaling molecules such as protein kinase C, arachidonic acid, and rho kinase, increase the sensitivity of vascular smooth muscle cells to contractile stimuli by inhibiting PP1M (Bradley et al., J. Physiol. (London) 385:437, 1987; Kitazawa et al., J Biol. Chem., 266:1708, 1991; Masuo et al., J Gen. Physiol., 104:265-2869, 1994; Cui Gong, et al., J. Biol. Chem., 267:21492-21498, 1992; Kimura, et al., Science, 273:245-248, 1996). Conversely, endogenous nitric oxide and related nitrovasodilators regulate blood pressure and cause vasodilation by activation of soluble guanylate cyclase, elevation of cyclic guanosine monophosphate (cGMP), and activation of cGMP-dependent protein kinase 1α (cGK1α), thus mediating the physiological relaxation of vascular smooth muscle in response to nitric oxide and cGMP.

Cyclic GMP-mediated vascular smooth muscle cell relaxation is characterized by both a reduction of intracellular calcium concentration and by cGMP-dependent activation of PP1M, which reduces the sensitivity of the contractile apparatus to intracellular calcium (Lincoln in Cyclic GMP: Biochemistry, Physiology and Pathophysiology (R.G. Landes Company, Austin, 1994) pp. 97-115; Lohmann et al., TIBS, 22: 307-312, 1997; Pfeifer, et al., EMBO J 17: 3045, 1998; Morgan et al., J. Physiol., 357:539, 1984; Lincoln et al., FASEB J. 7: 328, 1993; Cornwell et al., J. Biol. Chem. 264: 1146-1155, 1989; Wu et al., Biochem Biophys Res Commun, 220:658-663, 1996; Lee et al., J Biol. Chem., 272:5063-5068, 1997).

This invention provides a link between cGKIα and PP1M and features a novel assay for the identification of compounds that affect the contractile state of smooth muscle.

### Methods

### Colony-lift Filter Assay

Colonies were lifted onto NitroPure nitrocellulose filters (MSI, Westboro, Mass.) which were then submerged in liquid nitrogen for 15 seconds, placed on presoaked Whatman #5 paper (in Z buffer: 60 mM Na₂HPO₄.7H₂O, 40 mM NaH₂PO₄.H₂O, 10 mM KCl, 1 mM MgSO₄.7H₂O), 50 mM β-mercaptoethanol, pH 7.0, and incubated at 37° for up to 12 hours.

### Saphenous Vein Smooth Muscle Cell Culture

Human saphenous vein smooth muscle cells (passage≦3) were grown in DME to near-confluency. Cells were scraped into PBS, centrifuged at 1500 rpm for 6 minutes at 4° C., and resuspended in 0.5 ml of lysis buffer A (50 mM Tris Cl, pH 7.5, 7 mM MgCl₂, 2 mM EDTA, 2 mg/ml n-Dodecyl-B-maltoside, 0.4 mg/ml cholesteryl hemisuccinate, 0.6M NaCl, 10 mM Na Molybdate, 1 mM PMSF, 10 μg/ml chymostatin, 200 μg/ml aprotinin, 50 μg/ml leupeptin) and incubated for 1 hour at room temperature.

### GST-Fusion Protein Experiments

Lysates were centrifuged for 15 minutes at 4° C. in a microfuge, and the supernatant was incubated with 100 μl of GST or GST-fusion protein beads overnight, followed by washing in RIPA buffer containing 1% NP40, and boiling for 5 minutes in SDS sample buffer. Associated proteins were resolved by SDS-PAGE and transferred to nitrocellulose, which was blocked in PBS with 0.05% Tween 20 and 5% milk. Primary antibody incubation with rabbit polyclonal anti-human cGPK-CT (Upstate Biotechnology, Lake Placid, N.Y.) was carried out at 1:2000 dilution and rabbit polyclonal anti-MBS antibody (Berkeley Antibody Company, Richmond, Calif.) at 1:1600 dilution in blocking buffer for one hour at room temperature, followed by secondary antibody incubation with anti-rabbit horseradish peroxidase-conjugated IgG (Amersham Life Science, Arlington Heights, Ill.) at 1:500 in blocking buffer. The membranes were developed with ECL (Amersham Life Science, Arlington Heights, Ill.).

### Immunoprecipitation Experiments

Lysates from human saphenous vein smooth muscle cells were prepared as described herein, precleared with protein A sepharose beads, and then incubated overnight with 5 μg of non-immune goat serum, rabbit polyclonal anti-MBS or goat polyclonal anti-cGK, followed by harvest with protein A beads. SDS-PAGE and immunoblots were performed according to standard procedures and as described herein. For MBS co-immunoprecipitation experiments, a cell pellet, prepared as described above, was resuspended in lysis buffer B (25 mM Tris Cl, pH 7.5, 5 mM MgCl₂, 2.5 mM EDTA, 1% Triton X 100 and protease inhibitors as in Buffer A). The lysate was incubated for 1 hour at room temperature, centrifuged in a microfuge for 5 seconds, and the supernatant precleared with 12.5 μg rabbit IgG followed by protein A beads. The precleared supernatant was incubated with either rabbit non-immune IgG, or rabbit polyclonal anti-MBS (Berkeley Antibody Company, Richmond, Calif.) overnight, followed by harvest with protein A beads. Equal amounts of rabbit nonimmune and anti-MBS antibodies were added and verified by Ponceau staining. SDS-PAGE and immunoblots were performed according to standard procedures and as described herein.

### Phosphatase Assay

Immunopellets, prepared as described herein, were washed and resuspended in phosphatase assay buffer A (20 mM MOPS, 20 mM glucose, 1 mM dithiothreitol, 1 mM theophylline, 1 mg/ml BSA, and 5 mM azide, pH 7.5). ³²P-labeled phosphorylase a (final concentration 10 μM; Dr. D. Brautigan, University of Virginia, Va.) in either buffer A alone, buffer A with 10 mM 8-bromo-cGMP, buffer A with 2 nM okadaic acid, or buffer A with 1 μM okadaic acid was added to the immunopellets. The reactions were incubated at 30° C. for 30 minutes and terminated with trichloroacetic acid. In separate experiments, ³²P-myosin light chains were used as substrate in place of phosphorylase a.

### Kinase Assays

In vitro cGK-mediated phosphorylations of the MBS immunopellet (prepared using lysis buffer A) or 10 μg of histone F2b were performed in kinase buffer, including 20 mM Tris pH 7.5, 10 mM Magnesium acetate, 20 μM ATP, 1 μM okadaic acid, 10 μM cGMP, 350 nM cGK1α, and 10 μCi ³²P-γ-ATP, for 15 minutes at 30° C. SDS-PAGE was performed as above. A constitutively active cGK (cGK-CA) in which amino acids 1-77, including the targeting leucine zipper domain and the autoinhibitory domain are removed by proteolysis (Monken et al., J. Biol. Chem. 255: 7067, 1980; Heil et al., Eur. J. Biochem. 168:117, 1987), was prepared by incubating 50 μg of bovine cGK1α (purified as in Lincoln et al., J. Biol. Chem. 252: 4269, 1977) with 1 μg trypsin for 3 minutes at 30° C. The reaction was terminated by the addition of 5 μg of soybean trypsin inhibitor. Image analysis of gel bands was performed using Scion Image software, and data are presented as mean+/−SE.

### Immunoflourescence

Human saphenous vein smooth muscle cells of passage 2-4 were grown on coverslips, washed with phosphate buffered saline (PBS), and fixed with 3% paraformaldehyde for 15 minutes at room temperature. Cells were then washed and permeabilized with 0.3% Triton X 100 in PBS with 10% donkey serum for 15 minutes at room temperature. To preserve stress fiber architecture prior to fixation, cells were washed on ice with PBS for 1 minute and permeabilized with 0.3% Triton X in 50 mM Tris pH 7.4 containing 0.5 mM PMSF, 1 μg/ml leupeptin, 1 μg/ml aprotinin and 1 μg/ml pepstatin on ice for 1 minute as described (Ishihara, et al, Biochem. Biophys. Res. Com., 159: 871-877, 1989; Brautigan et al., Methods in Enz. 159: 339, 1988; Cohen, Methods in Enz. 201:389, 1991). The cells were then washed with PBS and blocked with 10% donkey serum in PBS for 1 hour at 37° C., followed by washing with PBS. Primary antibody mixtures were rabbit polyclonal anti-MBS (1/125), or goat polyclonal anti-cGK (1/250). Secondary antibodies were donkey anti-rabbit IgG-conjugated Cy3 (Amersham Life Science) (1/800) and donkey anti-rabbit IgG-conjugated FITC (Chemicon International Inc.) (1/100). Following incubation with secondary antibody, the coverslips were washed with PBS and mounted in Slow Fade (Molecular Probes, Eugene, Oreg.).

### Assay

Smooth muscle cells may be obtained from tissue discards, obtained at the time of surgery, or from commercial sources. Complementary DNAs, corresponding to cyclic GMP-dependent protein kinase 1α (cGK1α) or the myosin binding subunit of myosin phosphatase (MBS) (Dr. M. Mendelsohn, New England Medical Center, Mass.), or fragments thereof, may be cloned into appropriate vectors by standard techniques. Alternatively, cGK1α or MBS may be obtained and purified from endogenous sources.

Protein levels may be ascertained by standard assays such as immunoblotting or immunoprecipitation. Endogenous cGK1α or MBS may be distinguished from cGK1α or MBS by, for example, epitope tagging the introduced receptor with myc or HA tags.

Cyclic GMP or analogs of cGMP, such as 8-Bromo-cyclic GMP, may be used at concentrations known to activate cGK1α. Nitrovasodilators, such as nitric oxide, made be used at concentrations known to mediate contractile state of smooth muscle. Concentrations of the test compounds may be approximately 10⁻¹² -10⁻⁵ M.

Interaction of cGK1α and MBS may be assayed by techniques such as the yeast two-hybrid system, co-immunoprecipitation, GST-fusion protein interaction, fluorescence spectroscopy, or any other protein interaction assay known in the art. The contractile state of smooth muscle may be measured by PP1M phosphatase activity, myosin light chain phosphorylation state, the tone of a vascular ring or strip in a vascular bioassay system, the length of individual smooth muscle cells, and related indices.

### EXAMPLE 1

### Interaction of Cyclic GMP-dependent Protein Kinase 1 α with the Myosin Binding Subunit of Myosin Phosphatase

### Full Length Cyclic GMP-dependent Protein Kinase 1 α

The yeast two-hybrid protein interaction system was used to identify potential binding partners of human cyclic GMP-dependent protein kinase 1α (cGK1α). The full-length coding region of cGK1α (Tamura et al., Hypertension, 27:552-557, 1996) was cloned in-frame into the Sal I site of the Gal4 DNA binding domain (GalDB) vector, PC97 (Chevray et al., Proc. Natl. Acad. Sci., 89:5789-5793, 1992; Vidal et al., Proc. Natl. Acad. Sci., 93:10315-10320,1996), to create the construct cGK-PC97. cGK-PC97 and a human activated T cell library (Clontech, Palo Alto, Calif.), cloned into a Gal4 DNA activating domain (GalAD) vector, PC86 (Dr. M. Vidal, Massachusetts General Hospital, MA), were transformed into Saccharomyces cerevisiae strain MaV103, using the lithium acetate method (Glatz et al., Nucleic Acids Res., 20:1425, 1992).

Polypeptides interacting with cGK-PC97 were detected by their ability to activate transcription of histidine (HIS3) and β-galactosidase (LacZ) reporter genes via reconstitution of the Gal4 transcription factor. MaV103 colonies capable of growing on media lacking histidine were assayed for β-galactosidase activity using the colony-lift filter assay, according to standard techniques and as described herein. PC86 plasmids (containing the activated T cell library) from His3+, LacZ+ colonies were isolated and the library cDNA insert sequenced. 2.5×10⁶ clones were screened from the T cell library.

Clone AL9 was found to transactivate both HIS3 and LacZ reporter genes when used in combination with cGK1α. Sequencing of clone AL9 revealed that it encoded the carboxy-terminal 181 amino acids of the myosin binding subunit (MBS) of myosin phosphatase (PP1M), which includes a leucine zipper domain (amino acids 1007-1028 of human MBS; Takahashi et al., Genomics, 44:150-152, 1997). MBS is the 130 kDa regulatory subunit of PP1M which confers specificity of PP1M for myosin light chain and is the site of regulation of PP1M by rho kinase (Alessi et al., Eur. J. Biochem., 210: 10231-35, 1992; Hirano et al., J. Biol. Chem. 272:3683, 1997; Johnson et al., Eur. J. Biochem., 239:317, 1996; Shimizu et al., J. Biol. Chem. 269:30407, 1994; Bradley et al., J. Physiol. (London) 385:437, 1987; Kitazawa et al., J. Biol. Chem., 266:1708, 1991; Masuo et al., J. Gen. Physiol., 104:265-2869, 1994; Gong et al., J. Biol. Chem., 267:21492-21498, 1992; Kimura et al., Science, 273:245-248,1996).

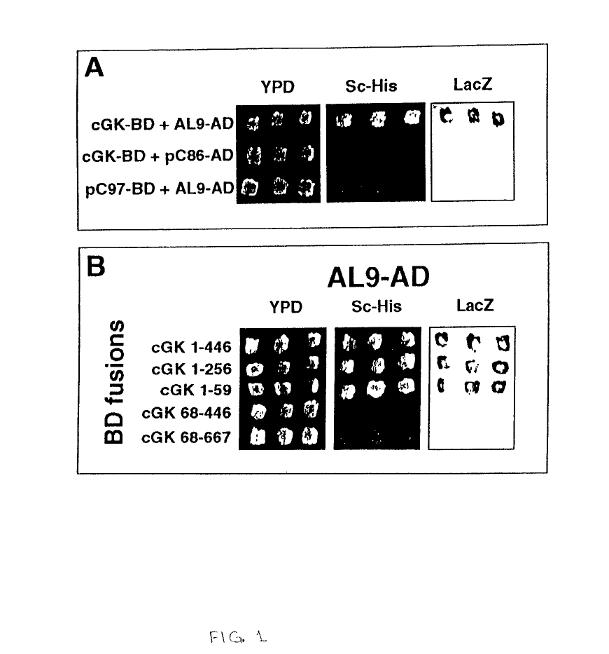

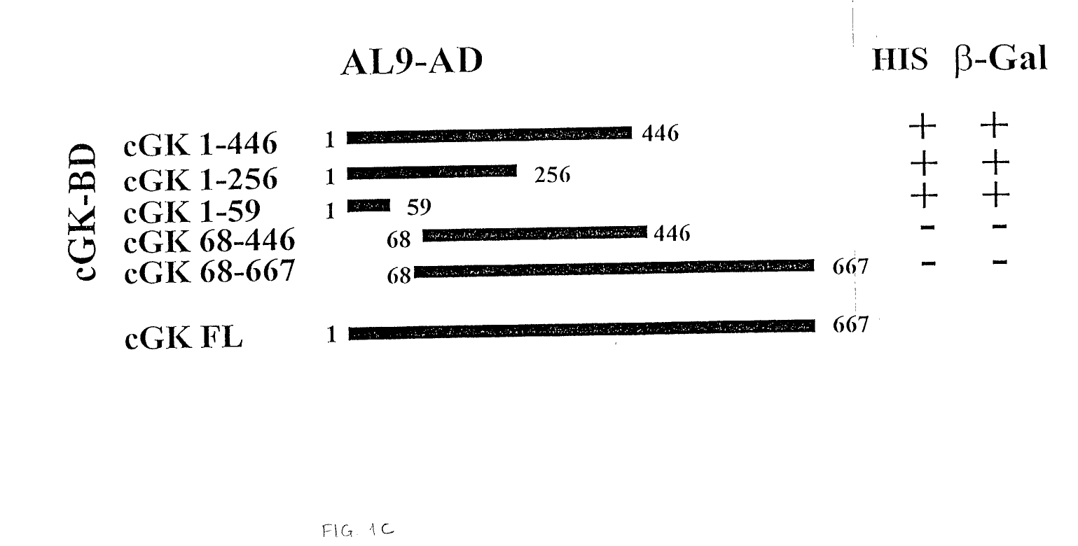

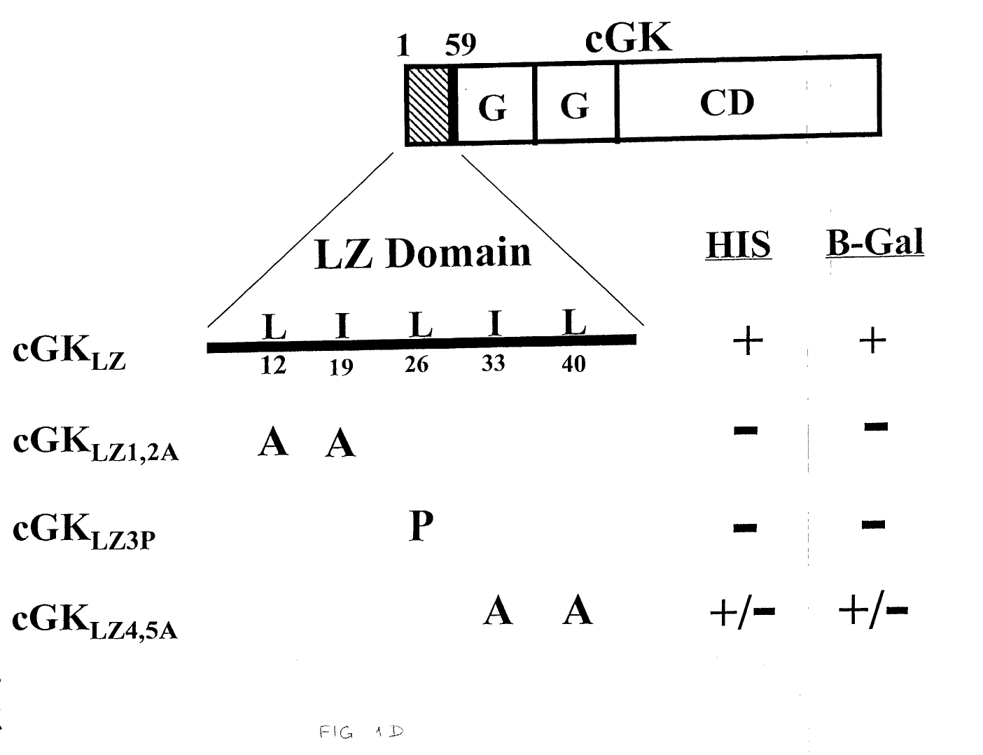

To verify the interaction of AL9 with cGK1α, individual plasmids were introduced into S. cerevisiae strain Y190 (Clontech, Palo Alto, Calif.) by the lithium acetate method and assayed for reporter gene activation, as above. Y190 was transformed with cGK1α/GalDB or GalDB alone, in combination with AL9/GalAD or GalAD alone, and plated on Sc-His (synthetic complete medium, minus histidine) and YPD (yeast extract, peptone, dextrose) plates. β-galactosidase activity assays, on colonies from the YPD plate, were also performed. All transformants were viable on the YPD plates but only yeast containing both AL9 and cGK1α sequences grew on Sc-His plates and showed LacZ activity (FIG. 1A). Thus, cotransformation of AL9 and cGK1α into S. cerevisiae confirmed the interaction of full-length cGK1α with the carboxy-terminal 181 amino acids of MBS.

### Truncation Mutants of Cyclic GMP-dependent Protein Kinase 1 α

Yeast two hybrid protein interaction assays, using truncation mutants of cGK1α, were carried out to define the cGK1α domain that interacts with MBS. N-terminal cGK1α truncation mutants were constructed in PC97 as follows: The Sac I cGK1α fragment was excised to yield a construct containing amino acids 1-446 (cGK1α₁₋₄₄₆); the Aat II cGK1α fragment was excised to yield a construct containing amino acids 1-256 (cGK1α₁₋₂₅₆); and the Sma I cGK1α fragment was excised to yield a construct containing amino acids 1-59 (cGK1α₁₋₅₉). Following restriction enzyme digestion, cGK1α was self-ligated in the PC97 vector to yield cGK1α/GalDB truncation mutants. To create cGK1α mutants corresponding to amino acids 68-446 (cGK1α₆₈₋₄₄₆) and 68-667 (cGK1α₆₈₋₆₆₇), Pst I sites were engineered at amino acids 4 and 68 with degenerate PCR primers. The resultant PCR product was ligated into the cGK1α sequence. The Pst I fragment was excised by restriction digestion, and the C-terminal cGK1α was then ligated to yield construct cGK 68-667 in the PC97 vector. The construct cGK 68-446 was made by similar methods. The reading frames of the resulting cGK1α/GalDB truncation mutants were verified by DNA sequencing and the constructs transformed into Y190 cells. Expression of the C-terminal constructs in PC97 was confirmed by immunoblotting the Y190 lysate using a rabbit polyclonal Gal4 DNA binding domain antibody (Upstate Biotechnology, Lake Placid, N.Y.).

Yeast strain Y190 was transformed with AL9/GalAD in combination with the cGK1α/GalDB truncation mutants and plated on YPD and Sc-His plates. Colony lift β-Galactosidase assays for the yeast colonies growing on the YPD plate were also performed. N-terminal cGK1α fragments of 446 amino acids (cGK1α₁₋₄₄₆), 256 amino acids (cGK1α₁₋₂₅₆), and 59 amino acids (cGK1α₁₋₅₉) all interacted with AL9 (FIG. 1B). In contrast, an internal fragment of cGK1α consisting of amino acids 68-446 (cGK1α₆₈₋₄₄₆), and a C-terminal cGK1α clone in which the first 67 amino acids were deleted (cGK1α₆₈₋₆₆₇), both failed to associate with AL9 (FIG. 1B). The results of FIG. 1B are summarized in FIG. 1C, which shows histidine (HIS) and β-Galactosidase (β-Gal) reporter gene activation for the cGK1α/GalDB truncations. Thus, the C-terminus of MBS (AL9) interacts with the N-terminus of cGK1α (cGK1α₁₋₅₉).

### Leucine/Isoleucine Mutants of cGK1α

The N-terminus of cGK1α is known to encode the leucine-isoleucine zipper-containing regulatory domain of cGK1α (Atkinson et al., Biochemistry 30:9387-9395, 1991; Landschulz et al., Science 240:1759-1764, 1988; Landschultz et al., Science 243: 1689, 1989; Harbury et al., Science 262: 1401, 1993). The N-terminal leucine-isoleucine zipper motif of cGK1α is located between amino acids 12 and 40 (Lincoln in Cyclic GMP: Biochemistry, Physiology and Pathophysiology (R. G. Landes Company, Austin, 1994) pp. 97-115; Lohmann et al., TIBS, 22: 307-312, 1997; Pfeifer, et al., EMBO J. 17: 3045, 1998; Tamura, et al., Hypertension, 27:552-557, 1996; Atkinson et al., Biochemistry 30:9387-9395, 1991; Landschulz et al., Science 240:1759-1764, 1988; Landschultz et al., Science 243: 1689, 1989; Harbury et al., Science 262: 1401, 1993). The C-terminus of MBS also contains a leucine zipper motif, located between residues 1007-1028 (Alessi et al., Eur. J. Biochem., 210: 1023-1035, 1992; Hirano et al., J. Biol. Chem. 272: 3683, 1997; Johnson, et al., Eur. J. Biochem., 239: 317, 1996; Shimizu, et al. J. Biol. Chem. 269: 30407, 1994). Leucine and leucine-isoleucine zipper motifs are helical heptad repeats known to mediate protein-protein interactions. Replacement of leucine residues with alanine, valine, or proline has been shown to abrogate leucine zipper-mediated binding. The importance of the leucine-isoleucine zipper of cGKIα for binding to MBS was tested using yeast two hybrid interaction assays.

Site-directed mutagenesis was employed to replace selected leucine or isoleucine residues in the N-terminus of cGKIα. cGK1α₁₋₅₉ was cloned into vector pcDNA3.1 (Invitrogen, Carlsbad, Calif.) using the method of Kunkel et al. (Kunkel et al., Methods in Enzymology, 154: 367-382, 1987). Three mutants of the leucine/isoleucine zipper of cGKIα were prepared: Leu₁₂ and Ile₁₉ to Ala (cGKL_{LZ1,2A}); Leu₂₆ to Pro (cGK_{LZ3P}); Ile₃₃ and Leu₄₀ to Ala (cGK_{LZ4,5A}). All mutants were confirmed by DNA sequencing, then subcloned into vector PC97 for expression in yeast. cGK_{Lz1,2A}, GK_{LZ3P}, and GK_{LZ4,5A} were each cotransformed with AL9 in yeast strain Y190 , and reporter gene activation assayed as described herein.

In yeast two-hybrid assays, cGK_{LZ4,5A} showed weak association with MBS, whereas cGK_{LZ1,2A} and cGK_{LZ3P} both failed to interact with MBS. The yeast two hybrid results for the leucine/isoleucine mutants are summarized in FIG. 1D. The binding of wild type cGKIα N-terminus (cGK_{LZ}) and mutants cGK_{LZ1,2A}, cGK_{LZ3P} and cGK_{LZ4,5A} to AL9, assayed by HIS and α-gal reporter activation in yeast, is shown on the right, where (+) indicates strong binding, (+/−) indicates weak binding, and (−) indicates no binding. G represents the cGMP binding site and CD the catalytic domain of cGKIα. These experiments show that the leucine-isoleucine zipper motif of cGKIα, in the N-terminal regulatory region, specifically mediates the interaction with the leucine zipper-containing C-terminal domain of MBS.

A model of the interaction of MBS and cGK1α is shown in FIG. 1E, where G indicates the cGMP binding site and CD the catalytic domain of cGK1α. The N-terminal leucine-isoleucine zipper in cGK1α interacts with the carboxyl terminal MBS leucine zipper domain. PP1M binds myosin light chain (MLC20) through the N-terminal ankyrin repeat region of MBS. PP1M also contains the catalytic subunit of PP1 (PP1_{c}) and a third subunit (M20).

### EXAMPLE 2

### Specific Interaction Between MBS and cGKIα in Human Vascular Smooth Muscle Cells

### GST-Fusion Protein Binding Studies

The interaction of cGK1α and MBS was also examined by GST-fusion protein binding studies (Atkinson et al., Biochemistry 30:9387-9395, 1991) using lysates from human vascular smooth muscle cells.

The 586 base pair cDNA insert from AL9, the 177 base pair N-terminal cGK1α truncation mutant, cGK1α₁₋₅₉, the N-terminal 274 base pairs of cGKIβ, and the N-terminal 816 base pairs of cGKII were cloned into pGEX (Pharmacia Biotech, Piscataway, N.J.). GST fusion proteins were expressed in bacterial strain BL21 (Stratagene, La Jolla, Calif.) according to standard techniques. The expression and concentration of the fusion proteins were confirmed by SDS-PAGE and Coomassie blue staining.

Lysates for experiments using GST-AL9 were derived from frozen saphenous vein which was homogenized in RIPA buffer, with protease inhibitors and 1% Triton X-100, at 4° C., followed by centrifugation at 3500 rpm for six minutes at 4° C. Lysates used for all other GST-fusion protein experiments were from cultured saphenous vein smooth muscle cells, prepared as described herein. GST-fusion protein interaction assays were performed, using the lysates, as described herein.

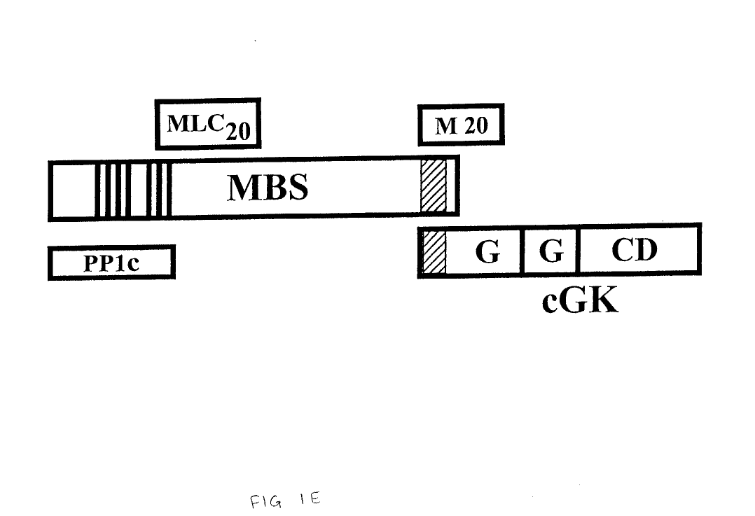

FIG. 2A is an immunoblot showing the interaction between AL9 and cGKIα in human vascular smooth muscle cell lysates. The lysates were incubated with glutathione agarose beads (lane 1), GST beads (lane 2), or GST-AL9 beads (lane 3) followed by SDS-PAGE and immunoblotting with anti-cGK antibody. GST-AL9, but not GST alone (negative control), bound cGK from human saphenous vein smooth muscle cell lysates. The 690 amino acid N-terminal half of MBS (MBS₁₋₆₉₀) showed no interaction with cGK in similar experiments. Conversely, GST-cGK₁₋₅₉ also specifically bound MBS from human vascular smooth muscle cell lysates (FIG. 2B).

Mammalian tissues contain two additional cGK isoforms, cGKIβ and cGKII, which share considerable sequence homology to cGKIα (Lincoln in Cyclic GMP: Biochemistry, Physiology and Pathophysiology, R. G. Landes Company, Austin, 1994, pp. 97-115.; Lohmann et al., TIBS, 22:307-312, 1997; Pfeiffer et al., EMBO J. 17: 3045, 1998), but differ substantially in their N-terminal domains from cGKIα. Peptides derived from cGK isoforms Iα, Iβ and II were tested for binding to MBS derived from human saphenous vein smooth muscle cell lysates (FIG. 2B). The lysates were incubated with GST beads (lane 1), GST-cGK1α₁₋₅₉ beads (lane 2), GST-cGK1β₁₋₉₂ beads (lane 3), and GST-cGKII-₁₋₂₇₂ beads (lane 4) and immunoblotted for MBS. One of two similar experiments is shown.

Neither GST-cGKIβ nor GST-cGKII bound MBS from vascular smooth muscle cell lysates, indicating that the interaction with MBS is specific to the Iα isoform of cGK. Yeast two hybrid assays also did not indicate any interaction between GST-cGKIβ or GST-cGKII and MBS.

Binding of the leucine/isoleucine mutants of cGKIα to MBS was also tested using GST-fusion protein assays. The leucine/isoleucine zipper mutants of cGKIα prepared as described above (cGK_{LZ1,2A}, cGK_{LZ3P}, and cGK_{LZ4,5A}) were subcloned into vector pGEX-4T-2 (Pharmacia Biotech, Piscataway, N.J.) for expression in bacteria. Equal amounts of GST, GST-cGK1α₁₋₅₉, GST-cGK_{LZ1,2A}, GST-cGK_{LZ3P}, and GST-cGK_{LZ4,5A} were incubated with saphenous vein smooth muscle cell lysates and GST pulldown experiments were performed as described herein.

None of the leucine zipper mutants interacted with MBS from vascular smooth muscle cell lysates in GST-fusion protein studies, confirming the yeast two hybrid data presented herein.

### Co-immunoprecipitation of MBS and cGK

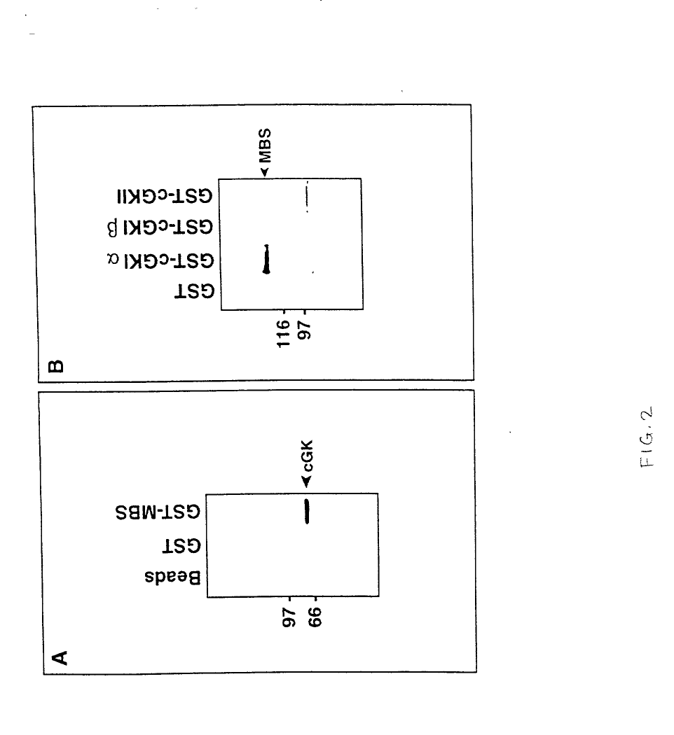

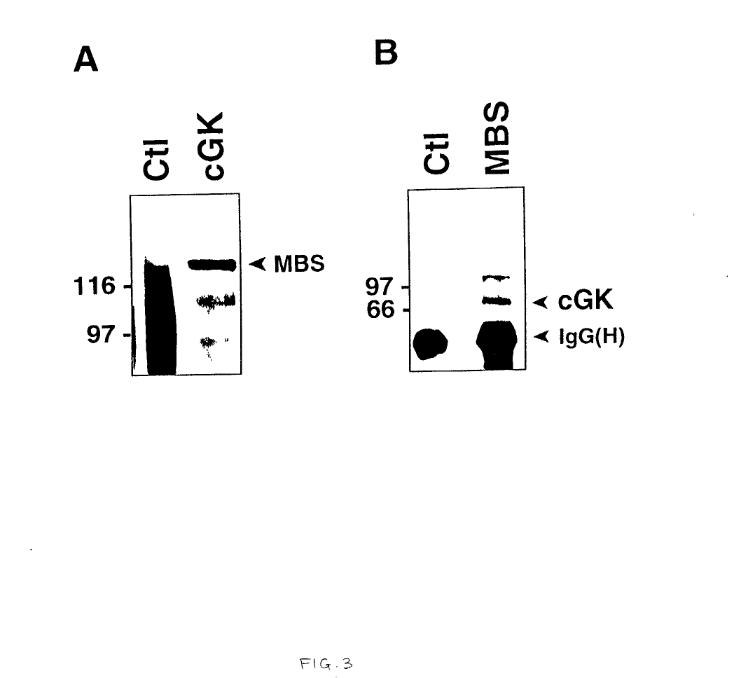

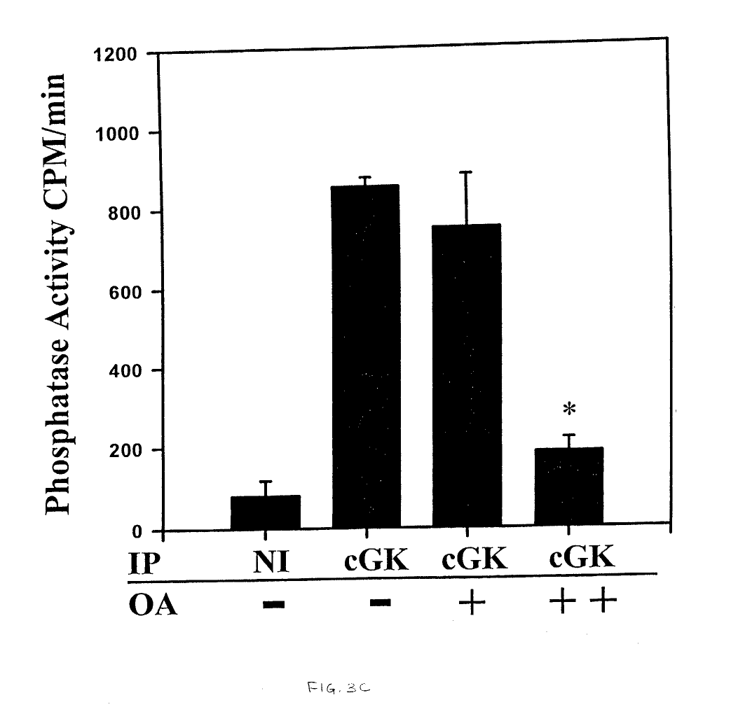

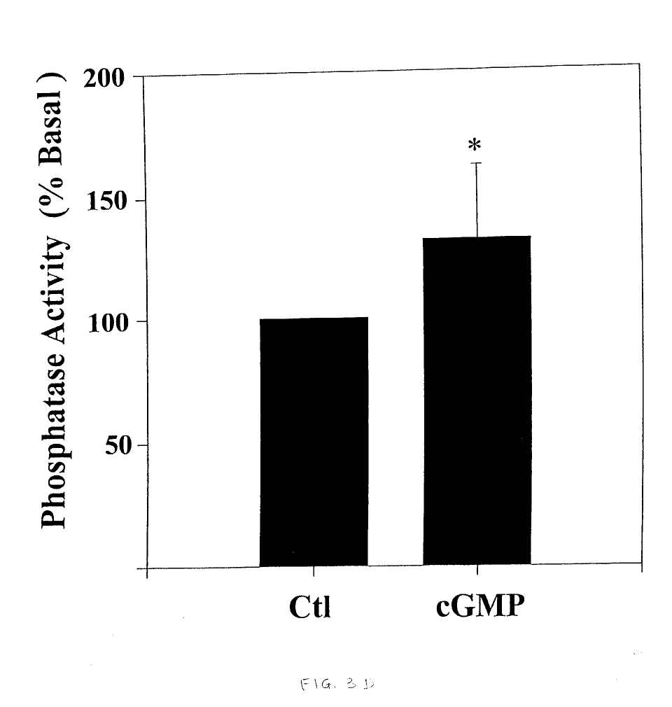

The interaction of cGKIα and MBS in vascular smooth muscle cells was also tested by immunoprecipitation experiments, as described herein. Lysates from cultured saphenous vein smooth muscle cells were immunoprecipitated with either nonimmune IgG or anti-cGKIα antibodies, resolved on SDS-PAGE, and immunoblotted for MBS (arrow) FIG. 3A. Immunoprecipitates of cGKIα from human vascular smooth muscle cell lysates contained MBS.

Lysates from saphenous vein smooth muscle cells were also immunoprecipitated with either nonimmune IgG, or anti-MBS antibodies, resolved by SDS-PAGE, and immunoblotted for cGK (arrow) (FIG. 3B). As in the anti-cGK immunoprecipitates, when MBS was immunoprecipitated, cGKIα was detected in the immunopellet.

### EXAMPLE 3

### Stoichiometry of the Binding of cGKIα and MBS

The stoichiometry of the binding of cGKIα and MBS was explored using fluorescence spectroscopy. Fluorescence spectroscopy was performed as previously described (MacMillan-Crow et al., Biochemistry 33:8035, 1994). Binding of labeled cGKIα to GST-MBS was specific and saturable, with a Kd of 62 nM. Linear transformation of the data (Gutfreund, “Ligand Binding,” in Enzymes: Physical Principles, (John Wiley & Sons, Inc., London: 1971), pp. 67-94) demonstrated that cGKIα, which exists as a dimer, binds MBS in a 1:1 molar ratio, indicating that each dimer of cGKIα binds a dimer of MBS.

### EXAMPLE 4

### Association of PP1M Phosphatase Activity with cGKIα

PP1M phosphatase activity in the cGKIα immunoprecipitates was measured against two known PP1M substrates, myosin light chain and phosphorylase a. cGKIα was immunoprecipitated and phosphatase activity was assayed in the immunopellets as described herein.

FIG. 3C is a bar graph showing the association of PP1M activity with cGKIα. NI refers to nonimmune IgG, cGK refers to anti-cGKIα immunopellets, and OA refers to okadaic acid, 2 nM (+) or 1 μM (++). One of two similar experiments is shown. Phosphatase activity was present in the cGKIα immunopellets and was only minimally inhibited by 2 nM okadaic acid (12+/−10%, P=NS, n=3). 1 μM okadaic acid, however, led to significant inhibition (79+/−2%, *=p<0.001 vs. untreated, n=3), characteristic of the effects of this inhibitor on PP1 phosphatases (Ishihara, et al., Biochem. Biophys. Res. Com., 159: 871-877, 1989; Brautigan et al., Methods in Enz. 159:339, 1988; Cohen, Methods in Enz. 201:389, 1991).

Since cGMP is known to augment PP1M activity in permeabilized smooth muscle (Bradley et al., J. Physiol. (London) 385:437, 1987; Kitazawa et al., J. Biol. Chem., 266:1708, 1991; Masuo et al., J. Gen. Physiol., 104:265-2869, 1994; Cui Gong, et al., J. Biol. Chem., 267:21492-21498, 1992; Kimura, et al., Science, 273:245-248, 1996), the nonhydrolyzable cGMP analog, 8-bromo-cGMP, was added to anti-MBS immunopellets to examine regulation of PP1M by cGMP/cGKIα. 8-bromo-cGMP significantly increased the level of PP1phosphatase activity (32+/−13%, n=5, p=<0.001) in the MBS immunopellet (FIG. 3D). Furthermore, in two separate experiments, the activation of PP1M by 8-bromo-cGMP was prevented by preincubation of the complex with the specific cGK inhibitor Rp-8-pCPT-cGMPS. These experiments demonstrate that cGKIα is complexed with fully functional PP1M phosphatase activity and that cGMP activates PP1 activity in the MBS immunopellets in a manner that requires cGKIα.

### EXAMPLE 5

### Phosphorylation of Proteins in the MBS Immunopellet by cGKIα

Phosphorylation studies were conducted to identify potential cGKIα substrates in the cGKIα-PP1M complex. Kinase assays, performed according to standard techniques and as described herein, demonstrated marked phosphorylation of MBS by cGKIα, as well as of four other proteins of molecular weights 72 KDa, 57 KDa, 42 KDa, and 20-26 KDa (arrowheads; FIG. 3E). The general kinase substrate, histone F2b, was used as a control for cGKIα kinase activity. Addition of cGMP and cGKIα (final concentration, 350 nM) to the anti-MBS immunopellets in the presence of γ[³²P]ATP led to markedly increased phosphorylation of the MBS itself (FIG. 3E).

Since the N-terminal domain of cGKIα mediates binding to MBS (FIGS. 1 and 2), and MBS is a substrate of cGKIα, the targeting of cGKIα to its substrate, MBS, by the N-terminal domain of cGKIα was examined.

Trypsin digestion of cGKIα removes the first 77 amino acids of the enzyme, including both the leucine-isoleucine zipper and auto-inhibitory domains, and results in a constitutively active cGKIα (cGK-CA) (Monken et al., J. Biol. Chem. 255:7067, 1980; Heil et al., Eur. J. Biochem 168:117,1987). In vitro MBS phosphorylation assays with constitutively active cGKIα (cGK-CA), fill length cGKIα (cGK-FL), or without cGK (Ctl), were performed. Either cGK-CA or full-length cGK1α were incubated with the MBS immunopellet or histone F2b in the presence of γ[³²P]ATP. Phosphorylation of MBS by cGK-CA was markedly reduced in comparison to phosphorylation of MBS by cGKIα (76+/−3%, P<0.003, n=3). However, both cGK-CA and cGKIα phosphorylated histone F2b to a similar extent (FIG. 3F). These data further demonstrate that MBS is a substrate for cGKIα, and that the N-terminal leucine-isoleucine zipper domain of cGKIα is important in targeting cGKIα to MBS.

### EXAMPLE 6

### Subcellular Localization of cGKIα and MBS in Human Vascular Smooth Muscle Cells

Double-labeling immunofluorescence and confocal microscopy were used to determine the sub-cellular localization of cGKIα and MBS in human vascular smooth muscle cells as described herein.

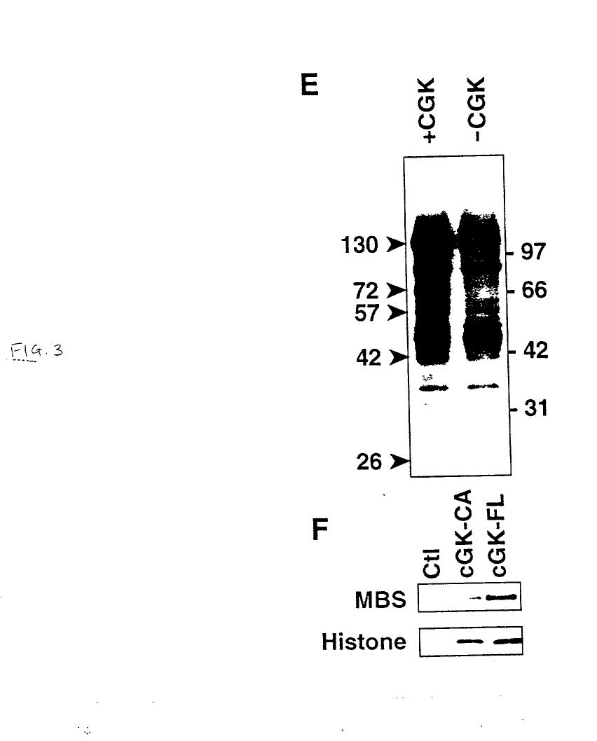

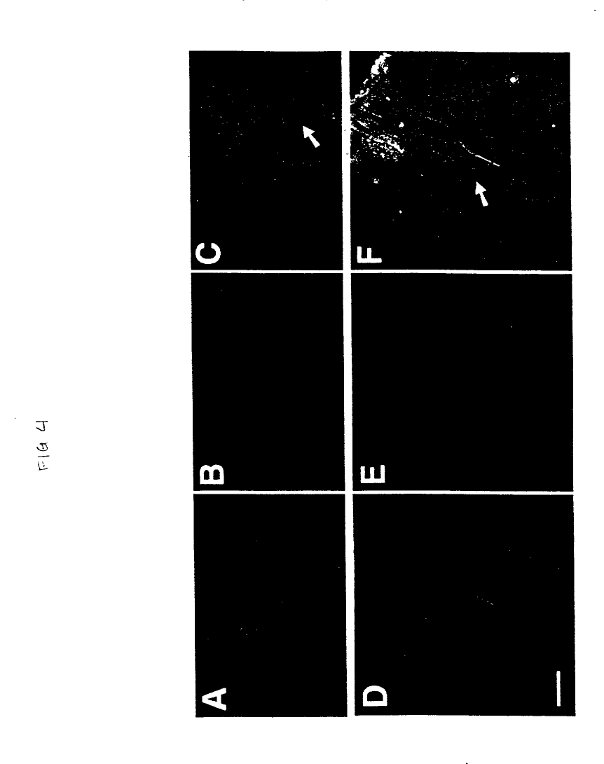

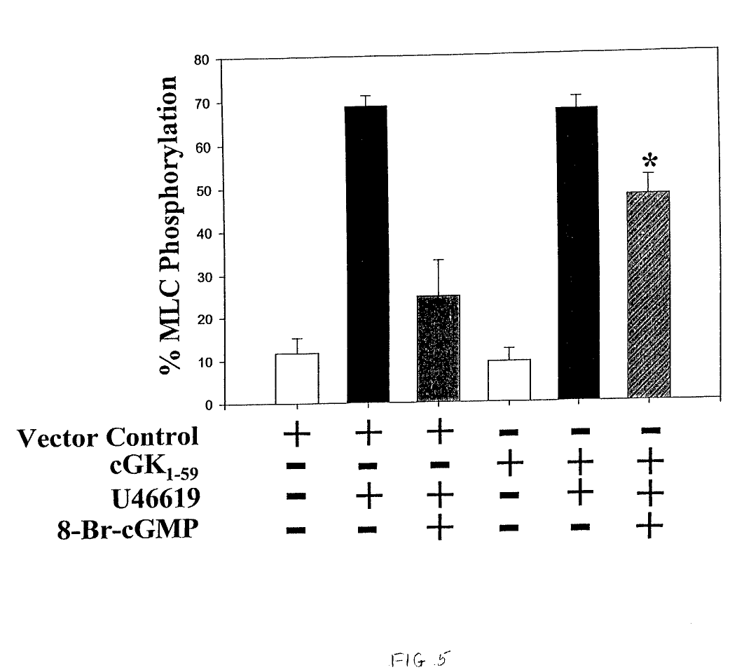

cGKIα and MBS colocalized consistently to two regions: a circumferential ring just below the plasma membrane (FIGS. 4A-4C), and to actin-myosin stress fibers in the vascular smooth muscle cells (FIGS. 4D-4F), where myosin light chain kinase and PP1M already are known to colocalize and regulate contraction (Somlyo et al., Nature, 372:231-236, 1994). Co-localization of MBS and cGKIα near the plasma membrane demonstrates that MBS is found in a site in addition to stress fibers in vascular smooth muscle cells and suggests this may position cGKIα near membrane protein substrates, such as G-protein coupled receptors, which have been shown recently to be regulated by cGKIα phosphorylation (Wang et al., Proc. Natl. Acad. Sci. U.S.A. 95:4888-4893, 1998). The localization of cGKIα to stress fibers (FIG. 4D-4F) has not been demonstrated previously, and shows further that the kinase is positioned in the cell where it may catalyze phosphorylation of MBS, and potentially several other proteins, to regulate vascular smooth muscle cell relaxation.

### EXAMPLE 7

### Effect of cGMP on Myosin Light Chain Phosphorylation Following Disruption of the cGK-MBS Interaction

In vascular smooth muscle cells, phosphorylation of the regulatory myosin light chain (MLC) is the key determinant of actomyosin ATPase activity and smooth muscle cell contraction (Somylo et al., Nature, 372: 231-236, 1994). The assay of MLC phosphorylation is an accepted measure of physiological contractile state in smooth muscles. Since MBS targets cGK1α to the smooth muscle cell contractile apparatus, and activation of cGK1α increases PP1M activity, the role of the cGK1α-MBS interaction in the regulation of smooth muscle cell contractile state by nitric oxide and cGMP was examined.

The extent of agonist-stimulated myosin light chain phosphorylation was quantified in intact vascular smooth muscle cells transfected with either vector alone or a plasmid expressing the cGK1α leucine/isoleucine zipper domain (CGK₁₋₅₉), to examine the effects of disrupting the cGK1α-MBS interaction on cGMP-mediated inhibition of myosin light chain phosphorylation. cGK1α₁₋₅₉ was cloned into the mammalian expression vector pCI (Promega, Madison, Wis.). Rat aortic smooth muscle cells of passage 6 through 10 were transfected with either vector alone (pCI) or cDNA for the leucine/isoleucine zipper peptide from cGK (cGK1α₁₋₅₉pCI) by electroporation. Cells were arrested in DME supplemented with insulin, ascorbic acid and transferrin, then stimulated with 2 μM of the thromboxane analogue U46619 for 1 minute in the absence or presence of 20 minute pretreatment with 1 mM 8-Br-cGMP. Following treatment, cells were precipitated with trichloroacetic acid, washed with acetone, and protein was subjected to glycerol-urea electrophoresis (Taylor et al., J. Biol Chem. 263, 14456, 1988). Myosin light chain phosphorylation state was quantified by immunoblotting the unphosphorylated and phosphorylated forms of myosin light chain with monoclonal antimyosin light chain antibody (Sigma, clone MY-21). Protein bands were analyzed by densitometry. Statistical analysis was performed using Student-Newman-Keuls method. Data represent the means+/−standard error of three separate experiments in duplicate.

The thromboxane analogue U46619 caused an increase in myosin light chain phosphorylation form 10+/−2% to 68+/−2% (P<0.001, n=3) in both vector control and cGK₁₋₅₉-transfected vascular smooth muscle cells (FIG. 5). In control-transfected vascular smooth muscle cells, 8-Br-cGMP inhibited U46619-mediated myosin light chain phosphorylation strongly (by 79+/−17%, P<0.001, n=3; FIG. 5). Expression of cGK₁₋₅₉, however, significantly disrupted 8-Br-cGMP-mediated inhibition of U46619-stimulated myosin light chain phosphorylation (from 79% to 35% inhibition, *P=0.001, n=3). Thus, disruption of the cGK1α-MBS interaction prevents cGMP-mediated dephosphorylation of myosin light chain, the central determinant of contractile state in intact vascular smooth muscle cells, and reverses the ability of cGMP to inhibit myosin light chain phosphorylation and mediate smooth muscle cell relaxation.

The invention, therefore, shows for the first time that cGK1α binds specifically to the MBS of the phosphatase PP1M via a leucine zipper interaction, which targets cGK1α to stress fibers to mediate smooth muscle cell relaxation and vasodilation in response to rises in intracellular cGMP. In addition, these studies demonstrate MBS is a substrate of cGK1α, cGMP stimulates PP1M activity in the cGK1α-MBS complex, and disruption of the cGK1α-MBS interaction impairs cGMP-mediated dephosphorylation of myosin light chain, the critical determinant of smooth muscle cell contractile state.

MBS, which contains N-terminal ankyrin repeats in addition to a C-terminal leucine zipper, is also complexed with the 37 Kd catalytic subunit of PP1M, the 20 Kd subunit of the phosphatase (M20), the regulatory MLC, and rho kinase. Thus MBS assembles a multi-enzyme complex, tethering a phosphatase and at least two distinct kinases with counter-regulatory effects on PP1M activity to the contractile apparatus to regulate smooth muscle contraction and relaxation.

### Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth, and follows in the scope of the appended claims.

What is claimed is:

1. A method of assaying whether a test compound affects the contractile state of smooth muscle cells, said method comprising:
(a) providing a system comprising
(i) cyclic GMP-dependent protein kinase or a fragment thereof, and
(ii) a myosin phosphatase myosin-binding subunit or a fragment thereof, under conditions in which said kinase can interact with said subunit;
(b) contacting said test compound with said system; and
(c) determining whether said test compound affects the interaction between said kinase and said subunit as an indication of the ability of said test compound to affect the contractile state of smooth muscle cells.

2. The method of claim 1, wherein said cyclic GMP-dependent protein kinase is full length cGK1α

3. The method of claim 1, wherein said cyclic GMP-dependent protein kinase is cGk 1-59.

4. The method of claim 1, wherein said cyclic GMP-dependent protein kinase is a leucine zipper mutant of said kinase.

5. The method of claim 1, wherein said myosin phosphatase myosin-binding subunit is full length.

6. The method of claim 1, wherein said myosin phosphatase myosin-binding subunit fragment is AL9.

7. The method of claim 1, wherein said smooth muscle cell is a vascular smooth muscle cell.

8. The method of claim 1, wherein said determining further comprises determining phosphatase activity.

9. The method of claim 1, wherein said determining further comprises determining the kinase activity of said kinase.

10. The method of claim 1, wherein said determining further comprises determining myosin light chain phosphorylation state.

11. The method of claim 1, wherein said determining further comprises determining vascular tone in a ring or strip bioassay.

12. The method of claim 1, wherein said determining further comprises determining vascular smooth muscle cell length.

13. The method of claim 1, wherein said determining further comprises determining the sub-cellular location of said kinase or said phosphatase.

14. The method of claim 1, wherein said determining is done in vitro.

15. The method of claim 1, wherein said determining is done in vivo.

16. The method of claim 1, wherein said determining is done using a yeast two-hybrid system.

17. The method of claim 1, wherein said determining is done by immunoprecipitation.

18. The method of claim 1, wherein said determining is done by GST-fusion protein interaction assay.

19. The method of claim 1, wherein said determining is done by fluorescence spectroscopy.

20. The method of claim 1, wherein said system further comprises a nitrovasodilator.

21. The method of claim 1, wherein said system further comprises a cGMP analogue.

22. The method of claim 20, wherein said nitrovasodilator is nitric oxide.

23. The method of claim 20, wherein said nitrovasodilator is a nitrate.

24. The method of claim 21, wherein said cGMP analogue is 8-bromo-cyclic GMP.

25. The method of claim 1, wherein said system further comprises a compound that alters cyclic GMP levels in a cell.

26. The method of claim 25, wherein said compound is a phosphodiesterase inhibitor.

27. The method of claim 26, wherein said phosphodiesterase inhibitor is Viagra™.

28. The method of claim 1, wherein said system further comprises a non-nitrate vasodilator.
